(19) United States
(12) Patent Application Publication
(10) Pub. No.: US 2024/0001038 A1
(43) Pub. Date: Jan. 4, 2024

(54) FAST DATA TRANSMISSION FEATURE FOR MEDICAL LOGGING DEVICE

(71) Applicant: Novo Nordisk A/S, Bagsvaerd (DK)

(72) Inventors: Per Einar Pontus Holm, Malmoe (SE); Jens Aage Munk, Oelstykke (DK)

(21) Appl. No.: 18/037,813

(22) PCT Filed: Nov. 23, 2021
(86) PCT No.: PCT/EP2021/082607

§ 371 Date: May 19, 2023

(30) Foreign Application Priority Data
Nov. 24, 2020 (EP) 20209435.5
Nov. 23, 2021 (WO) PCT/EP2021/082607

(51) Int. Cl. A61M 5/315; G16H 20/17; G16H 40/67

(52) U.S. Cl. CPC A61M 5/31568; A61M 5/31548; G16H 20/17; G16H 40/67; A61M 2205/3584; H04W 4/38

(57) ABSTRACT

A drug delivery device comprising electronic circuitry comprising sensor means adapted to capture a property value related to the dose amount of drug expelled from the reservoir by the expelling means a during an expelling event, storage means adapted to store a plurality of property values to create a data log, and transmission means for wireless transfer of the data log to an external device at a low speed. The transmission means is adapted to listen for and receive a scan request packet whereby the transmission means is operated to wireless transfer of the data log to an external device at a higher speed.

The present invention generally relates to methods and devices for wirelessly communicating a dynamic data log from a data generating device, e.g. to medical devices for which the generation, collecting and storing of data are relevant. In specific embodiments the invention relates to devices and methods for capturing and transmitting drug delivery dose data in a reliable and user-friendly way.

### BACKGROUND OF THE INVENTION

In the disclosure of the present invention reference is mostly made to drug delivery devices comprising a threaded piston rod driven by a rotating drive member, such devices being used e.g. in the treatment of diabetes by delivery of insulin, however, this is only an exemplary use of the present invention as it may be implemented in any given technical field in which the transfer of a dynamic data log is relevant, e.g. for medical devices in general in which drugs are administered or in which physiological data is measured and logged.

Drug injection devices have greatly improved the lives of patients who must self-administer drugs and biological agents. Drug injection devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. To make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the notebook system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Correspondingly, some proposed drug delivery devices integrate this monitoring/acquisition mechanism into the device itself, e.g. as disclosed in US 2009/0318865, WO 2010/052275 and WO 2016/110592, these devices being of the durable type, whereas WO 2015/071354 discloses a disposable drug delivery device provided with dose logging circuitry.

However, most devices of today are without it. Addressing this problem, many solutions have been proposed which would help a user to generate, collect and distribute data indicative of the use of a given medical device. For example, WO 2013/120776 describes an electronic supplementary device (or add-on device) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. A further external device for a pen device is shown in WO 2014/161952, the external device being designed to determine dose sizes based on detection of movement of a magnetic member incorporated in the pen device.

Although the above-described logging devices in general are provided with a display allowing logged dose data to be displayed, it may be desirable to transfer dose data to an external device, e.g. a smartphone as carried by many drug delivery device users, this allowing the dose data to be displayed on a much larger display and to be further processed and used for e.g. analysis and recommendations. Such an arrangement would also allow the display on the logging device to be dispensed with. WO 2019/170828 discloses a medical device with dose logging capability in which data is transferred to an external device by priority-optimized low speed data transfer in order to reduce power consumption.

Having regard to the above, it is an object of the present invention to provide devices and methods allowing efficient and cost-effective wireless transfer of a dynamic data log from a data generating device, e.g. from a drug delivery device or from a physiological sensor device with logging capabilities to an external device such as a smartphone.

### DISCLOSURE OF THE INVENTION

In the disclosure of the present invention, embodiments and aspects will be described which will address one or more of the above objects or which will address objects apparent from the below disclosure as well as from the description of exemplary embodiments. In the context of the present disclosure the term BLE is used as a short-form for “Bluetooth™ Low Energy” or “Bluetooth™ LE”.

When a drug delivery device is provided with a dose logging functionality but with no display, it relies on transmission of dose log data to an external device for subsequent viewing, e.g. a user's smartphone. In order to reduce power consumption data transmission may take place with a low speed allowing a complete log to be received by the external over time. As mentioned above, the most recent dose logs may be prioritized in such a transmission set-up. To speed up transmission of the most recent dose log to the external device, the transmission means may be set up to transmit data at a higher speed for a given amount of time after a dose amount has been delivered, this allowing the user to almost immediately view the most recent dose log on e.g. his or hers smartphone. However, in case the external device is not in the vicinity of the drug delivery device during one or more drug delivery events, transfer of the dose log may rely primarily on low speed data transmission during periods in which the drug delivery device is not in use but in vicinity of the external device. In this way data can be transmitted “in the background” in a slow but steady way allowing the entire log to be transmitted over e.g. a number of hours.

In case the user does not need to always have a fully updated dose log available to view, the above-described set-up may represent an acceptable compromise between low power consumption of the dose logging circuitry and availability of dose log data for the user. Also, some users may not be interested in the dose log data at all.

However, the present invention is based on the realization that in some special situations, there may be a need for fast transmission of an entire dose log in a short period of time, e.g. within seconds instead of e.g. the 30 minutes that may be needed for transmission of a complete large dose log at the low transmission speed. In case the drug delivery device is provided with the ability to transmit data at a higher speed subsequent to a drug delivery event, this may only be sufficient to transmit the most recent dose log data, just as the drug delivery device may be empty and it thus may not be possible to initiate fast data transmission. One specific example would be when the user/patient comes to the doctor's office and the doctor has to get access to the dose log data in order to evaluate the patient's treatment. In some cases it may be possible to transmit the dose log from the patient's smartphone to the doctor's computer, however, for a number of patients the dose log will only be stored in the drug delivery device.

Thus, in a first aspect of the invention a drug delivery device is provided, comprising a drug reservoir, drug expelling means comprising dose setting means allowing a user to set a dose amount of drug to be expelled, and electronic circuitry adapted to create a data log related to expelled dose amounts of drug. The electronic circuitry comprises sensor means adapted to capture a property value related to the dose amount of drug expelled from the reservoir by the expelling means during an expelling event, storage means adapted to store a plurality of property values to create the data log, and transmission means for wireless transfer of the data log to an external device at a sleep-mode data transmission speed. The electronic circuitry is adapted to run an at least partially implemented BLE protocol stack configured to receive a scan request packet, and application software adapted to create a data log related to expelled dose amounts of drug. The BLE protocol stack is adapted to notify the application software when a scan request packet is received from an external scanner device, the application software being adapted to perform a predefined action depending on the source address of the external scanner. In the present context the term drug delivery device also covers a drug delivery assembly comprising an add-on dose logging device mounted on a drug delivery device per se.

By this arrangement a drug delivery device is provided adapted to transmit a dose log to an external device in a slow but power-efficient way yet allows the dose log to be transmitted faster in situations when needed. The BLE protocol stack may be implemented cost-effectively without the components for creating and maintaining a wireless two-way connection with an external device as in a traditional BLE implementation.

The drug delivery device may e.g. be adapted for subcutaneous delivery of a drug formulation, or it may be adapted for generating an aerosol of drug to be inhaled. The term property value indicates the “raw” data captured by the sensor means, e.g. the amount of rotation of an indicator member. As the captured values are related to the dose amounts of drug expelled from the reservoir by the expelling means during an expelling event in a known way, the captured values allow the dose amounts to be calculated, either in the drug delivery device or subsequently in the external device.

In an exemplary embodiment, when the source address corresponds to a predefined address, the predefined action comprises operating the transmission means to full or partly wireless transfer of the data log at a fast-mode data transmission speed, the fast-mode data transmission speed being higher than the sleep-mode data transmission speed.

The transmission means may be configured to transfer the data log using the BLE protocol in advertising mode, the advertising mode comprising a scannable mode allowing the transmission means to listen for and receive the scan request packet.

The electronic circuitry may be adapted to determine when an end-of-life condition has been met, such that when the end-of-life condition has been met, the transmission means is operated in an end-of-life state in which it listens for and is adapted to receive the scan signal. In this way power consumption can be further lowered.

In an exemplary embodiment the end-of-life condition is a given total amount of drug having been expelled and the corresponding property values captured by the sensor means. The drug delivery device may be prefilled with a predetermined amount of drug contained in the reservoir, with the drug expelling means adapted to enter a device end-of-life state in which no dose amount can be expelled and thus no property values can be captured by the sensor means, e.g. a mechanical member may have been moved into an axial stop position. In such an arrangement the end-of-life condition can be set up to be met before the drug expelling means enters the device end-of-life state, this allowing the scannable mode to be activated when deemed necessary having regard to the expected use scenario for the given drug delivery device.

The transmission means may be configured to transfer the data log using a BLE protocol in advertising mode, the advertising mode comprising a non-scannable and a scannable mode, the scannable mode allowing the transmission means to listen for and receive the scan request packet, wherein the transmission means switches from the non-scannable mode to the scannable mode when the end-of-life condition has been met.

In an exemplary embodiment the transmission means is operated at a dose-mode data transmission speed for a given amount of time after the sensor means has captured a property value related to a dose amount of drug expelled from a reservoir by the expelling means, the dose-mode data transmission speed being higher than the sleep-mode data transmission speed.

The transmission means may be adapted to listen for and receive a scan request packet from a predefined address which includes a specific trigger information that will trigger the transmission means to transfer of the data log to the external device at the fast-mode data transmission speed. In this way the transmission means cannot be triggered to enter fast-mode by a random external device.

The transmission means may further be adapted to listen for and receive a scan request packet from one of a plurality of predefined addresses which includes at least one of a specific trigger information that will trigger the transmission means to transfer of the data log fully or partly to the external device at the fast-mode data transmission speed. In this way the actual portion of the data log that is transmitted can be tailored to a given predefined address.

In a further aspect of the invention a method for wirelessly communicating a data log from a data generating device is provided, comprising the steps of: using a BLE protocol stack in advertising scannable mode: transmitting the data log to an external device at a sleep-mode data transmission speed, the scannable mode allowing the transmission means to listen for and receive a scan request packet, detecting a scan request packet, and after having detected a scan request packet: transmitting the data log to an external device at a fast-mode data transmission speed, the fast-mode data transmission speed being higher than the sleep-mode data transmission speed.

The BLE protocol stack may be implemented and operated cost-effectively without the components for creating and maintaining a wireless two-way connection with an external device as in a traditional BLE implementation.

The method for wirelessly transmitting a data log may comprise the initial steps (i.e. executed before the above-described steps) of: using a BLE protocol stack in advertising non-scannable mode: transmitting the data log to an external device at the sleep-mode data transmission speed, detecting an end-of-life condition for the data generating device, and after having detected the end-of-life condition for the data generating device: using the BLE protocol stack in advertising scannable mode.

The scannable mode may allow the transmission means to listen for and receive a scan request packet from a predefined address including a specific trigger information, such that the data log is transmitted only when a scan request packet from a predefined address including a specific trigger is received.

In an exemplary embodiment the method for wirelessly transmitting a data log, the data generating device comprises a reservoir containing an initial amount of drug and drug expelling means allowing a user to set a dose amount of drug to be expelled, wherein the end-of-life condition is detected when a given amount of drug has been expelled from the reservoir.

In a general aspect of the invention a data generating medical device is provided, comprising electronic circuitry adapted to create a data log related to a medical property, comprising sensor means adapted to capture a property value related to a medical property, storage means adapted to store a plurality of property values to create the data log, and transmission means for wireless transfer of the data log to an external device at a sleep-mode data transmission speed, wherein the transmission means is adapted to listen for and receive a scan request packet whereby the transmission means is operated to wireless transfer of the data log to an external device at a fast-mode data transmission speed, the fast-mode data transmission speed being higher than the sleep-mode data transmission speed.

The sensor device may be an external device adapted to be mounted e.g. on a skin surface and adapted to measure and log a physiological parameter such as blood glucose values, O₂ saturation, ECG, skin temperature or blood pressure. The sensor means may e.g. be in the form of a percutaneous electrochemical sensor adapted for BG determination, or a non-invasive sensor such as a PPG sensor adapted for determination of O₂ saturation or blood pressure. Alternatively, the sensor device may be in the form of a device adapted to be implanted, e.g. a pacemaker adapted to measure and log electrocardiographic values.

As also disclosed above, the data generating medical device may be provided with a reservoir containing a drug, as well as drug expelling means allowing a user to set a dose amount of drug to be expelled, wherein the sensor means is adapted to capture a property value related to the dose amount of drug expelled from the reservoir by the expelling means during an expelling event.

In the context of the present application a number of functional and structural terms are used for components and functions of the electronic circuitry, e.g. transmission and storage means, however, these are only used to describe functional aspects of the present invention, the functions and structures typically being provided by integrated circuitry hardware in combination with layers of firm- and software.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone or drugs for haemophilia treatment.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIG. 2 is an exploded view of a dose sensing module adapted for incorporation in a drug delivery device of the type shown in FIG. 1A,

FIG. 3 is a perspective longitudinal section view of the dose sensing module of FIG. 2,

FIG. 4A shows a further drug delivery device,

FIG. 4B shows a flexible sheet with electronic circuitry,

FIG. 5 shows a yet further drug delivery device, and

FIG. 6 shows an add-on device mounted on a drug delivery device of the type shown in FIG. 1A.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

The present invention addresses the general issue of providing easy and power-efficient wireless transfer of a dynamic data log from a data generating device to an external device.

Correspondingly, a data transmission set-up for a data generating medical device in the form of a drug delivery device is provided, comprising a drug reservoir, drug expelling means comprising dose setting means allowing a user to set a dose amount of drug to be expelled, and electronic circuitry adapted to create a data log related to expelled dose amounts of drug. The electronic circuitry comprises sensor means adapted to capture a property value related to the dose amount of drug expelled from a reservoir by the expelling means during an expelling event, storage means adapted to store a plurality of property values to create the data log, and transmission means for wireless transfer of the data log to an external device at a sleep-mode data transmission speed.

According to an aspect of the invention, the transmission means is adapted to receive a scan signal whereby the transmission means is operated to wireless transfer of the data log to an external device at a fast-mode data transmission speed, the fast-mode data transmission speed being higher than the sleep-mode data transmission speed.

In a first exemplary embodiment timely, seamless, and cost-effective transfer from the data generating medical device to the external device is accomplished using a custom BLE radio chip. By removing the receiver part of the radio, the size and complexity of the radio chip can be significantly reduced and thus the cost. Such a radio chip may be incorporated in a drug delivery pen device with dose logging capabilities, this allowing for secure, easy and cost-effective wireless transfer of dose log data from the pen device to e.g. a mobile device such as a smartphone or a tablet computer.

With such a set-up, there is no handshake so the external device cannot query for e.g. the specific data it may lack due to previously non-received data. The data generating device must thus continuously transfer the whole log.

In the exemplary embodiment radio communication is based on the BLE standard, but in order to reduce complexity the device only acts as a so-called advertising device. This has the advantage of reducing both hardware and software complexity. There is no need to include the whole BLE protocol stack, and there is no need for a receiver. The advertising is of the type non-connectable and non-scan-able undirected advertising packet, referred to as ADV NON-CON IND in the BLE specification.

However, in the exemplary embodiment a radio that also have receiver capabilities is used. More specifically, the receiver can be used in a BLE advertising mode called scannable advertising (ADV SCAN IND PDU). In this mode the advertiser listens for scan requests from a scanner device and can respond with additional information in a scan response. The handshake can be handled purely by hard-ware, but the application can be notified if a scan has taken place together with the Bluetooth® address of the scanning device. When the drug delivery device firmware receives a scan request from a specific scanner it starts advertising more frequently.

In the exemplary embodiment the scan feature is implemented in the device firmware in the following way:

Scannable advertising: The fact that in the exemplary embodiment the chip used has a receiver can be utilized for debugging purposes by sending some of the packets as scannable packets. The scan response message is sent with a package as defined below. The following scan response packets are defined:

Header scan response

| 0 | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|---|
| 1E | FF | CB | 04 | 03 | | | | TH | TL | BH | BL | sw | Pin | | | | Dh | dl | | | | | | | | | | | | |

| BLE header | Typ | State | Temp | Bat- | sw | pin | boot | tap | Acc- |
|---|---|---|---|---|---|---|---|---|---|
| | | | | tLvl | | | | | Dose |

The Temp, BattLvl, sw, and pin (encoder pad state) fields are the same as above (but now available even if there are entries in the log).

State: Bit 0-5 of the state field contains the device state as follows

| Code | State | Description |
|---|---|---|
| 0 | IDLE | Idle mode, advertising headers every |
| | | 8^{th} second |
| 1 | DOSING | Encoder is moving |
| 2 | BLIND | Dose is detected and more encoder |
| | | input is ignored for a while |
| | | (debugging) |
| 3 | ACTIVE | Fast dose log advertising for 5 min |
| 4 | SILENT | No radio traffic until a dose (debug) |
| 5 | BEDTIME | Received command to hibernate |
| 6 | DREAM | Received command to hibernate with |
| | | RAM retention |

Bit 5—Universal mode enabled
Bit 6—Long recording mode enabled
Bit 7—An active scanner is detected

Boot: Number of resets, starting at 1, increments for each hibernation wakeup.

Tap: Total number of tap/drops detected

AccDose: Accumulated dose is the total number of clicks dosed from the device.

Encoder trace response packet: When the raw encoder trace packet is sent scannable the response packet is just another encoder trace packet.

Scanner detection: The Software Development Kit used in the exemplary embodiment provides the possibility to detect that a scannable advertisement is scanned by an active scanner and to retrieve the BDADDR of this scanner. This can be utilized by reacting on specific scanner addresses where a certain address can trigger a certain functionality.

In order to make it possible to use this feature the provider of an exemplary radio chip (Dialog semiconductor) has added a means in the API (Application Programming Interface) for the application software to receive signals when the device is scanned by an active scanner.

The following table gives the exemplary implementation:

Scanners with BDADDR FF:BE:FF:82:00:nn or FF:FF:FF:82:00:nn are recognized. The command code (nn) is interpreted as follows:

FB—High speed data transmission

FC—Universal On-set TX power to 0 dBm

FD—Universal Off-set TX power to protocol levels (−12 dBm normally)

FE—Start hibernating (an OTPed device goes to full hibernation, a non OTPed goes to hibernation with full RAM retention)

FF—Long recording started and buffer cleared

When the BLE circuitry recognizes e.g. the scannable address FF:BE:FF:82:00:FB, it will immediately shift to high speed data transmission and the full dose log can be transmitted within some seconds.

When operating the radio transmission means in the BLE scannable advertising mode, the power consumption is somewhat higher than when operating the radio transmission means in the BLE non-scannable advertising mode.

Although in a given implementation this higher level of power consumption would be acceptable, in the exemplary embodiment the transmission means can be operated in an initial first non-listening state and an activated second state in which the transmission means listen for and can receive a scan request packet when an end-of-life state has been determined.

A “real” end-of-life state could be linked to the mechanical end-of-life state for the drug delivery device per se, i.e. it is no longer possible for the user to expel an amount of drug from the device, however, this may require some kind of electro-mechanical switch arrangement interacting with the electronic circuitry, this adding to cost and complexity of the device.

Alternatively, an “assumed” end-of-life state could be detected in the electronic domain. For example, a given pre-filled drug delivery device is provided to the user with a guaranteed amount of drug, e.g. 300 IU of an insulin formulation, however, due to manufacturing tolerances it will be necessary to supply drug delivery devices which in most cases will contain and be able to expel more than the guaranteed amount of drug, e.g. 305 IU, which most users indeed will know and take advantage of.

Correspondingly, an assumed end-of-life state could be detected when the dose logging circuitry has registered that a total of e.g. 300 UI of an insulin drug formulation has been expelled, this providing that the radio transmission means shifts from the non-listening to the listening operation state. Indeed, in many cases the assumed and the real end-of-life state will be reached at the same time, e.g. 290 UI have been expelled and the user sets and expels a final dose of 15 UI.

In the above-described exemplary embodiment a specific implementation of a BLE radio is used, which is “self-contained” and can be implemented very cost-effectively. This said, it may be necessary to supply a special computer dongle, e.g. to the doctor, by which this BLE feature can be evoked. This may not be possible with a standard USB BLE dongle.

Alternatively, a user accessible sensor (e.g. mechanical or optical) could achieve the same function, however, due to cost, space and physical arrangement this may not be attractive for a given implementation of a dose logging and data transmission functionality in a given drug delivery device.

After having described an exemplary embodiment of a data transmission set-up, as well as some variations thereof, a number of drug delivery devices suitable for incorporation of the above-described transmission set-up will be described.

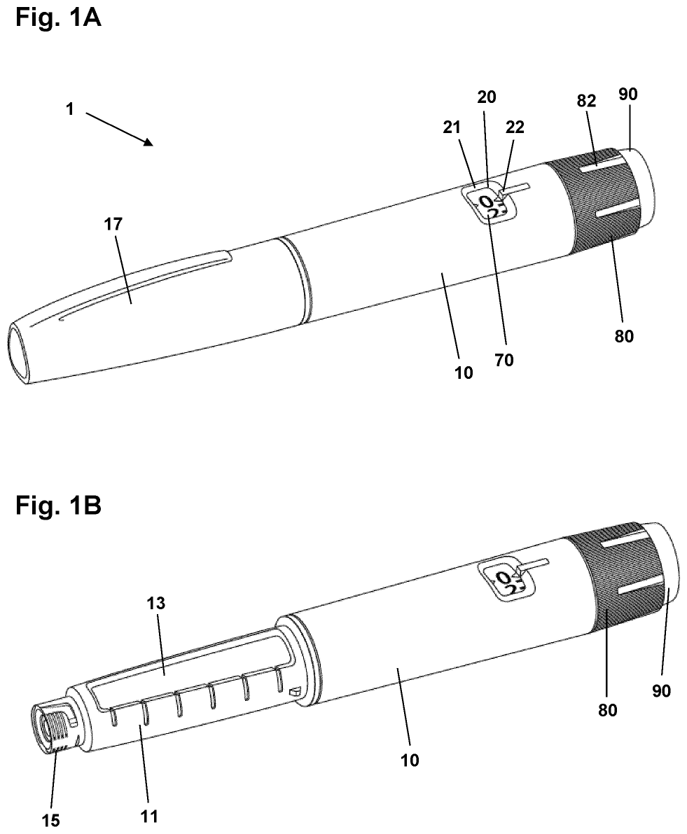

Before turning to embodiments of the present invention per se, an example of a prefilled drug delivery will be described, such a device providing the basis for an exemplary embodiment of the present invention. Although the pen-formed drug delivery device 1 shown in FIGS. 1A and 1B may represent a “generic” drug delivery device, the actually shown device is a FlexTouch® prefilled drug delivery pen as manufactured and sold by Novo Nordisk A/S, Bagsværd, Denmark.

The pen device 1 comprises a cap part 17 and a main part having a proximal body or drive assembly portion with a housing 10 in which a drug expelling mechanism is arranged or integrated, and a distal cartridge holder portion in which a drug-filled transparent cartridge 13 with a distal needle-penetrable septum is arranged and retained in place by a non-removable cartridge holder attached to the proximal portion, the cartridge holder having openings allowing a portion of the cartridge to be inspected as well as distal coupling means 15 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 80 with a number of axially oriented grooves 82 serves to manually set a desired dose of drug shown in display window 20 and which can then be expelled when the button 90 is actuated. The window is in the form of an opening in the housing surrounded by a chamfered edge portion 21 and a dose pointer 22, the window allowing a portion of a helically rotatable indicator member 70 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. A detailed description of the dose setting and expelling mechanism utilized in the FlexTouch® prefilled drug delivery pen can be found in e.g. WO 2019/057911. In summary, the mechanism will provide that during dose expelling the threaded piston rod is rotated an amount corresponding to the set dose, the rotation providing that the piston rod is axially advanced through a correspondingly threaded non-rotational nut element.

Alternatively the expelling mechanism may be fully manual in which case the dose member and the actuation button moves proximally during dose setting corresponding to the set dose size, and then is moved distally by the user to expel the set dose, e.g. as in a FlexPen® manufactured and sold by Novo Nordisk A/S.

Although FIG. 1A shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

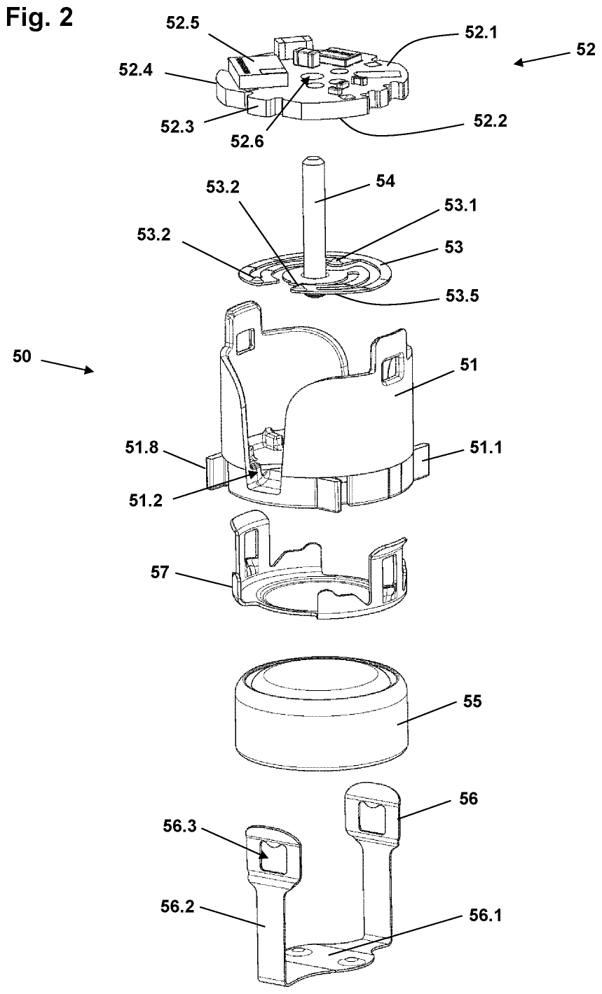

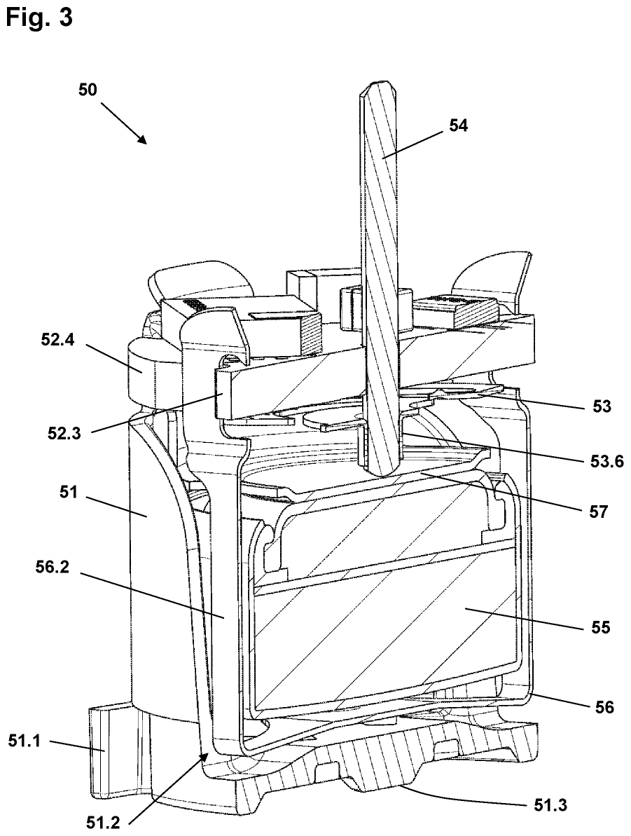

With reference to FIGS. 2 and 3 a rotary sensor module adapted to be incorporated in a drug delivery pen device of the above-described type, i.e. comprising a drug-filled cartridge with an axially moveable piston as well as a rotatable piston rod arranged to engage and axially move the piston to thereby expel drug from the cartridge, the amount of rotation of the piston rod being correlated with the amount of axial displacement. As will be apparent from the below description, the module is adapted to be arranged inside the cartridge proximally of the piston and with the piston rod non-rotationally engaging a wiper component. Such a module can be incorporated in a given pen device of this type with only minimal design modifications.

FIG. 2 is an exploded view highlighting the individual elements of a sensor module 50. The sensor module 50 comprises a first sensor part in the form of a PCB assembly 52 with a rigid support sheet 52.4 having a proximal surface 52.1 carrying various electronic components 52.5, including a processor, and a distal surface 52.2 carrying a plurality of electrically conductive sensor areas (not visible). The support sheet 52.4 has an overall circular periphery, but is provided with several notches, some of which resulting in a pair of diametrically opposite radial protrusions 52.3. Furthermore, the support sheet 52.4 has a central through-going bore 52.6.

The first sensor part is complemented by a second sensor part in the form of a wiper 53 being fixedly mounted to a piston rod connector 54 to ensure joint rotation therewith. The piston rod connector 54 extends axially through the through-going bore 52.6 and is adapted for press-fit engagement with a cavity in a distal end portion of a piston rod. This provides for a joint movement of the piston rod and the piston rod connector 54. The wiper 53 comprises one ground contact 53.1 and two code contacts 53.2 arranged on respective flexible arms 53.5 and adapted to galvanically connect with the electrically conductive sensor areas on the distal surface 52.2 of the support sheet 52.4, as described in more detail below. Notably, the ground contact 53.1 and the code contacts 53.2 are all proximally directed.

The two sensor parts, forming a rotary encoder system, are accommodated in a module housing 51 which also accommodates a power source in the form of a battery 55, a retainer 56 also functioning as a positive battery connector, and a rigid (negative) battery connector 57. The retainer 56 has a transversal support surface 56.1 for carrying the battery 55 and two axially extending opposite retainer arms 56.2. Each retainer arm 56.2 is provided with a proximal cutout 56.3 shaped to receive one of the radial protrusions 52.3, thereby rotationally interlocking the retainer 56 and the PCB assembly 52 and axially restricting the support sheet 52.4. The module housing 51 has a pair of diametrically opposite side openings 51.2 shaped to receive the retainer arms 56.2 so as to rotationally interlock, or at least substantially rotationally interlock, the retainer 56 and the module housing 51, and a plurality of anti-rotation tabs 51.1 spaced apart along its circumference, each anti-rotation tab 51.1 comprising a contact surface 51.8 for interaction with an interior surface of a cartridge wall. The PCB assembly 52 is thus at least substantially rotationally locked with respect to the module housing 51, which in turn is rotationally frictionally fitted in the pen device cartridge, which is rotationally fixed in the cartridge holder. The PCB assembly 52 is thereby at least substantially rotationally fixed with respect to the pen device housing and accordingly suitable as reference component for measuring angular displacements of the piston rod.

FIG. 3 is a perspective longitudinal section view of the sensor module 50 in an assembled state. As can be seen the piston rod connector 54 extends through the through-going bore 52.6 in the support sheet 52.4 and is press-fitted with a sleeve 53.6 on the wiper 53. The module housing 51 has a foot 51.3 which rests against the cartridge piston. Furthermore, the figure shows the position of the retainer arms 56.2 in the side openings 51.2 and the arrangement of the radial protrusions 52.3 in the cut-outs 56.3. During a dose expelling action with the module incorporated in a given injection device, the rotation of the piston rod is transferred to the piston rod connector 54 and further on to the wiper 53. The ground contact 53.1 and the code contacts 53.2 thus sweep the sensor areas of the distal surface 52.2 which remains, at least substantially, rotationally stationary due to the engagement between the radial protrusions 52.3 and the cut-outs 56, the fitting of the retainer arms 56.2 in the side openings 51.2, the frictional interface between the foot 51.3 and the piston 22, and the frictional interface between the antirotation tabs 51.1 and the cartridge wall.

The dose logging module 50 is described in greater detail in EP application 19201824.0.

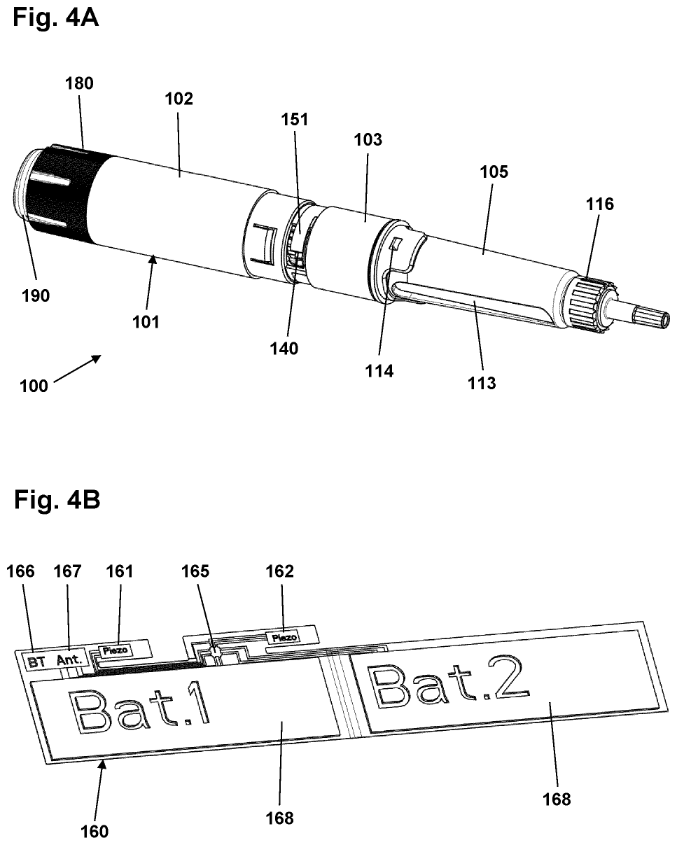

With reference to FIGS. 4A and 4B a further embodiment of a drug delivery device with integrated dose logging circuitry will be described.

The pen device 100 in FIG. 4A comprises a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion. The cartridge holder comprises openings allowing a portion of the cartridge to be inspected, distal coupling means allowing a needle assembly 116 to be releasably mounted as well as proximal coupling means in the form of two opposed protrusions 114 allowing a cap (not shown) to be releasably mounted covering the cartridge holder. In the shown embodiment the housing comprises a proximal housing portion 102 and a distal housing portion 103 which in a fully assembled state of the pen device is fixedly connected to each other via an intermediate tubular housing portion (not shown) covering the shown flexible arm 150 (see below), thereby forming a unitary housing. The cartridge is provided with a piston driven by a piston rod forming part of the expelling mechanism and may for example contain an insulin, GLP-1 or growth hormone formulation. A proximal-most rotatable dose setting member 180 serves to manually set a desired dose of drug and which can then be expelled when the button 190 is actuated. The expelling mechanism comprises a helically rotatable scale drum member with a plurality of indicia in the form of dose size numerals printed thereon, the dose size number corresponding to the currently set dose size being shown in a display opening (not seen in FIG. 3A). Depending on the type of expelling mechanism embodied in the drug delivery device, the expelling mechanism may comprise a spring as in the shown embodiment which is strained during dose setting and then released to drive the piston rod when the release button is actuated. Alternatively, the expelling mechanism may be fully manual in which case the dose member and the actuation button may be arranged to move proximally during dose setting corresponding to the set dose size, and then to be moved distally by the user to expel the set dose, e.g. as in a FlexPen® manufactured and sold by Novo Nordisk A/S.

Although FIG. 4A shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

The expelling mechanism incorporated in pen device 100 comprises a ring-formed piston rod drive element and an actuator member 140 in the form of a rotatable component that rotates together with the piston rod drive element during expelling of a dose of drug, the actuator member 140 thereby experiencing unidirectional rotational movement relative to an indicator structure fixedly disposed within the housing 101. In the shown embodiment the indicator structure is in the form of a pair of opposed circumferentially arranged deflectable flexible arms 151 each engaging the actuator member.

The actuator member 140 is in the form of a toothed wheel having a plurality of axially oriented ridges protruding radially outwards and being spaced circumferentially and equidistantly. Each ridge is formed with a gradually rising leading side and a sharply dropping trailing side. In the shown embodiment 24 ridges are spaced with angular steps of 15 degrees. Between any two neighbouring ridges a groove is formed.

Each of the deflectable arms 151 includes at its free end a tip portion with a radially inwards pointing first surface which is angled to be generally parallel with a gradually rising side of a ridge. Each tip portion further has a second opposed surface which is angled to be generally parallel with the sharply dropping side of a ridge. The radially inwards pointing first surface of the tip portions is configured to ride over consecutive ridges as the actuator member 140 rotates relative to the deflectable arms so that the tip portions of the first and second deflectable arm remain in intimate contact with the outer contour of the actuator member 140 as the latter rotates. The free end of a flexible arm 151 is biased slightly inwards when the tip portion is seated in a groove, the biasing force increasing when the free end of the arm is lifted outwards by the ridge formations as the actuator member rotates.

In the shown embodiment, the tip portions of the deflectable arms are located approximately 178 degrees apart so that, as the actuator member 140 rotates, the first deflectable arm will experience cooperation with a particular first ridge slightly before the second deflectable arm will experience cooperation with a ridge arranged diametrically opposite from the first protrusion. This arrangement is described in greater detail in EP application 17205309 hereby incorporated by reference. Alternatively, a single arm design may be used.

To monitor operation of the device by electronic means, electronic circuitry 160 is disposed in or on the device 100 for registering events associated with operations performed by the device, i.e. expelling of a set dose of drug. In the shown embodiment of FIG. 4B the electronic circuitry 160 is in the form of a flexible sheet on which is formed and mounted input means adapted to be actuated, directly or indirectly, by movement of the indicator structure(s), a processor and memory 165, wireless transmission means 166 with antenna 167, and an energy source 168, wherein the processor is adapted to determine on the basis of measured values from the input means a rotational position and/or a rotational movement of the actuator member 140 to thereby calculate the size of an expelled dose of drug. The flexible sheet is adapted to be mounted on housing parts of the pen device by e.g. adhesive means, the nature of the flexible sheet allowing it to be mounted also on curved surfaces.

In the shown embodiment the input means is active transducers in the form of piezoelectric sensors 161, 162 adapted to be mounted onto the flexible arms 151 and thereby generating an output as the flexible arms are moved by the rotating actuator member 140. Although not incorporated in the shown embodiment, the electronic circuitry may in other embodiments further include a display so as to offer a visible read-out of information related to registered events. In the shown embodiment energy is provided by two electric cells 168.

One or more of the above-described components may be printed onto the flexible sheet, e.g. the piezoelectric sensors, a display, the antenna and the energy source. Other components, e.g. the processor and associated memory as well as a BLE radio chip may be surface mounted on the flexible sheet.

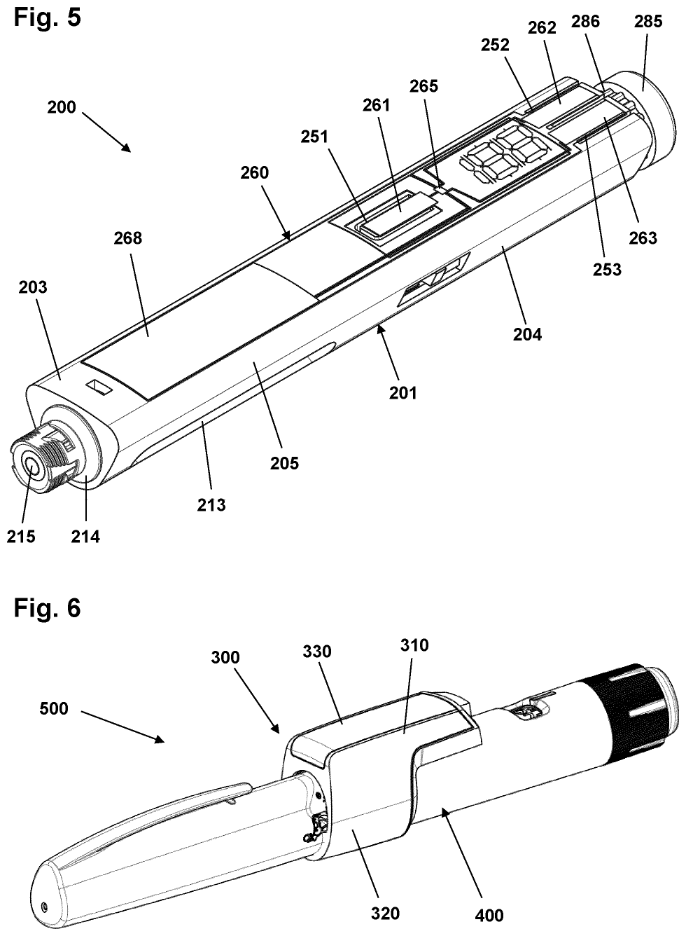

Turning to FIG. 5 a further pen device 200 incorporating electronic circuitry for the generation of a dynamic dose log will be described. The pen device 100 of FIG. 4A could be considered a traditional drug delivery device provided with electronic circuitry for the creation and transmission of a dose log, the pen device having a traditional user interface and being operated by a user in a traditional way, i.e. setting a dose size while observing a mechanical scale drum. In contrast, the pen device 200 is provided with a digital display replacing the traditional scale drum.

More specifically, the pen device 200 comprises a cylindrical housing 201 having a slightly curved information display surface 203 and a more conventionally curved opposing surface 204. The device is shown without a covering foil label, this allowing the electronic circuitry to be seen. The housing accommodates a drug containing cartridge 213, which has been inserted through an opening at a distal end thereof. The cartridge, which is closed at its distal end by a penetrable self-sealing septum 215 and at its proximal end by a slidable piston (not visible), is arranged in the distal cartridge holder portion 205 of the housing, being snapped to a proximal interior surface of the housing 201 by a snap coupling formed as part of the cartridge needle mount member 214 serving as an attachment interface for an injection needle unit (not shown). The housing is provided with a longitudinal window 206 for inspection of the cartridge contents and further accommodates both a dose setting mechanism and a drug expelling mechanism. The dose setting and expelling mechanism may be of any suitable design, e.g. a spring-driven design as shown, albeit without a scale drum. In the shown embodiment dose setting and dose release is performed using a combined dose setting and dose release member 285, i.e. the combined member is adapted to both rotate relative to the housing 201 during dose setting and to be moved axially to release a set dose.

As in the above-described embodiment, the expelling mechanism comprises an actuator member in the form of a rotatable component that rotates together with the piston rod drive element during expelling of a dose of drug, the actuator member thereby experiencing unidirectional rotational movement relative to an indicator structure fixedly disposed within the housing 201. In the shown embodiment the indicator structure is in the form of an axially arranged deflectable flexible arm 150 engaging the actuator member.

The combined dose setting and release member 285 extends into the housing 201 from a proximal end thereof. The combined member 285 comprises a cylindrical main body which is rotatable about a longitudinal axis of the housing. An axially grooved smaller-diameter actuator collar 286 is provided just distally of the main body and extends into the housing. The grooves have a spacing of 15 degrees and serve as actuators for dose setting input means, each groove corresponding to an increment of one dose unit, i.e. typically 1 IU of insulin.

In the housing 201 central portion some wall material has been removed to provide the abovementioned radially deflectable flexible dose expelling arm 250, and in a proximal portion wall material has been removed to provide first and second radially deflectable dose setting arms 251, 252, the latter being actuated by the grooved actuator collar 286. As described in greater detail in application EP2017/077850 the two dose setting arms allow incremental up/down rotation of the combined member 285 to be determined, this in turn being used to control the display to show the presently set dose size.

To monitor operation of the device by electronic means, electronic circuitry 260 is disposed on the device 200 for registering events associated with operations performed by the device, i.e. expelling of a set dose of drug. In the shown embodiment the electronic circuitry 260 is in the form of a flexible sheet on which is formed and mounted input means adapted to be actuated by movement of the indicator structures 250, 251, 252, a processor with memory and wireless transmission means 265, a display 269 and an energy source 268, wherein the processor is adapted to determine on the basis of measured values from the input means a rotational position and/or a rotational movement of the actuator member to thereby calculate the size of an expelled dose of drug. The flexible sheet is adapted to be mounted on the curved housing surface 203 of the pen device by e.g. adhesive means.

In the shown embodiment the input means is active transducers in the form of piezoelectric sensors 261, 262, 263 adapted to be mounted onto the flexible arms 251, 252, 253 and thereby generating an output as the flexible arms are moved by the rotating actuator member respectively the dose setting actuator collar 286.

One or more of the above-described components may be printed onto the flexible sheet, e.g. the piezoelectric sensors, the display, an antenna and the energy source in the form of an electric cell. Other components, e.g. the processor and associated memory as well as a BLE radio chip may be surface mounted on the flexible sheet.

A further type of a drug delivery device comprising integrated dose logging circuitry is in the form of a traditional manual (i.e. non-spring-driven) drug delivery device in which the dose setting and actuation button will extend axially from the device as a dose is being set, the dose logging circuitry being arranged in the dose setting button and comprising e.g. a traditional rotary encoder adapted to register rotation during dose setting and/or dose expelling. A specific example of such a device is sold and manufactured by Novo Nordisk A/S as the NovoPen® 6, a pen device provided with wireless NFC transmission means allowing dose log data to be transferred to an external device, however, the device may be modified to use a BLE radio implementing the above-described transmission set-up. NovoPen® 6 is provided with a display, however, this feature could alternatively be dispensed with.

Turning to FIG. 6 a drug delivery assembly 500 comprising an add-on dose logging device 300 mounted on a drug delivery pen device 400 of the spring-driven type is shown, the add-on device incorporating electronic circuitry for the generation of a dynamic dose log when mounted on the pen device. In the present context the device represents a “generic” drug delivery device providing a specific example of a device in combination with which embodiments of the present invention can be used.

The logging module 300 comprises a body portion 310 and a ring-formed portion 320 allowing the add-on device to be mounted on a generally cylindrical pen device. The body portion comprises electronic circuitry and sensor means allowing a property to be detected representing an amount of drug being expelled from the cartridge, as well as an optional display 330 for displaying data to a user. The ring portion comprises coupling means allowing the add-on device to be securely and correctly mounted on the pen body. The electronic circuitry and the sensor means may in part be arranged in the ring portion.

The pen device comprises an indicator element with a magnet rotating together therewith during expelling of a dose of drug, the magnet being configured to generate a spatial magnetic field which relative to the sensor means varies corresponding to the spatial position and orientation of the magnet. The add-on device comprises sensor means adapted to measure a magnetic field as well as processor means configured to determine based on measured values rotational movement and/or positions of the indicator element based on which a dose log can be created. An exemplary embodiment of both the add-on device and the pen device is described in greater detail WO 2014/161952 which is hereby incorporated by reference. Additionally, the shown add-on device 300 is provided with wireless transmission means allowing dose log data to be transferred to an external device using the above-described transmission set-up.

A further example of an add-on dose logging device adapted to be mounted on a drug delivery pen device of the spring-driven type is shown is shown in WO 2019/057911, hereby incorporated by reference.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

In the above disclosure aspects of the present invention has described based on implementation in a drug delivery device of the pen type typically used to inject drugs having a blood glucose controlling effect, e.g. human insulin and analogues thereof as well as non-insulins such as GLP-1 and analogues thereof, as well as other types of drug, e.g. growth hormone or drugs for haemophilia treatment. Alternatively, the drug delivery device may be in the form of a body-worn drug infusion pump for e.g. insulin formulations.

However, these are only exemplary implementations. For example, aspects of the present invention may be implemented in a sensor device adapted to be mounted e.g. on a skin surface and adapted to measure and log a physiological parameter such as blood glucose values or skin temperatures. Alternatively, the sensor device may be in the form of a device adapted to be implanted, e.g. a pacemaker adapted to measure and log electrocardiographic values.

1. A drug delivery device comprising:
a drug reservoir,
drug expelling structure comprising dose setting structure allowing a user to set a dose amount of drug to be expelled,
electronic circuitry adapted to create a data log related to expelled dose amounts of drug, comprising:
sensor structure adapted to capture a property value related to the dose amount of drug expelled from the reservoir by the expelling structure during an expelling event,
storage structure adapted to store a plurality of property values to create the data log, and
transmission structure for wireless transfer of the data log to an external device at a sleep-mode data transmission speed,
the electronic circuitry being adapted to run:
an at least partially implemented BLE protocol stack configured to receive a scan request packet, and
application software adapted to create a data log related to expelled dose amounts of drug, wherein the BLE protocol stack is adapted to notify the application software when a scan request packet is received from an external scanner device, the application software being adapted to perform a predefined action depending on the source address of the external scanner.

2. A drug delivery device as in claim 1, wherein:
when the source address corresponds to a predefined address, the predefined action comprises operating the transmission structure to full or partial wireless transfer of the data log at a fast-mode data transmission speed, the fast-mode data transmission speed being higher than the sleep-mode data transmission speed.

3. A drug delivery device as in claim 1, wherein:
the transmission structure is configured to transfer the data log using the BLE protocol in advertising mode, the advertising mode comprising a scannable mode allowing the transmission structure to listen for and receive the scan request packet.

4. A drug delivery device as in claim 1, wherein:
the electronic circuitry is adapted to determine when an end-of-life condition has been met, and
when the end-of-life condition has been met, the transmission structure is operated in an end-of-life state in which it listens for and is adapted to receive the scan request packet.

5. A drug delivery device as in claim 4, wherein the end-of-life condition is a given total amount of drug having been expelled and the corresponding property values captured by the sensor means.

6. A drug delivery device as in claim 5, wherein:
the drug delivery device is prefilled with a predetermined amount of drug contained in the reservoir,
the drug expelling structure is adapted to enter a device end-of-life state in which no dose amount can be expelled and thus no property values can be captured by the sensor means, and
the end-of-life condition is met before the drug expelling structure enters the device end-of-life state.

7. A drug delivery device as in claim 3, wherein:
the transmission structure is configured to transfer the data log using a BLE protocol stack in advertising mode, the advertising mode comprising a non-scannable and a scannable mode, the scannable mode allowing the transmission structure to listen for and receive the scan request packet, and
the transmission structure switches from the non-scannable mode to the scannable mode when the end-of-life condition has been met.

8. A drug delivery device as in claim 1, wherein:
the transmission structure is operated at a dose-mode data transmission speed for a given amount of time after the sensor structure has captured a property value related to a dose amount of drug expelled from a reservoir by the expelling means, the dose-mode data transmission speed being higher than the sleep-mode data transmission speed.

9. A drug delivery device as in claim 1, wherein:
the transmission structure is adapted to listen for and receive a scan request packet from one of a plurality of predefined source addresses which includes at least one of a specific trigger information that will trigger the transmission structure to fully or partly transfer of the data log to the external device at the fast-mode data transmission speed.

10. A drug delivery device as claim 1, wherein:
the BLE protocol stack is implemented without the components for creating and maintaining a wireless two-way connection with an external device.

11. A method for wirelessly transmitting a data log from a data generating device, comprising the steps of:
using a BLE protocol stack in advertising scannable mode: transmitting the data log to an external device at a sleep-mode data transmission speed, the advertising scannable mode allowing the transmission structure to listen for and receive a scan request packet,
detecting a scan request packet,
after having detected a scan request packet: using the BLE protocol stack in advertising scannable mode to transmit the data log to an external device at a fast-mode data transmission speed, the fast-mode data transmission speed being higher than the sleep-mode data transmission speed.

12. A method for wirelessly transmitting a data log as in claim 11, comprising the initial steps of:
using a BLE protocol stack in advertising non-scannable mode: transmitting the data log to an external device at the sleep-mode data transmission speed,
detecting an end-of-life condition for the data generating device,
after having detected the end-of-life condition for the data generating device: using the BLE protocol stack in advertising scannable mode.

13. A method for wirelessly transmitting a data log as in claim 12, wherein:
the data generating device comprises a reservoir containing an initial amount of drug and drug expelling structure allowing a user to set a dose amount of drug to be expelled, and
the end-of-life condition is detected when a given amount of drug has been expelled from the reservoir.

14. A method for wirelessly transmitting a data log as in claim 11, wherein:
the scannable mode allows the transmission structure to listen for and receive the scan request packet from a predefined address including a specific trigger information, and
the data log is transmitted when a scan request packet from a predefined address including a specific trigger is received.

15. A method for wirelessly transmitting a data log as in claim 11, wherein:
the BLE protocol stack is implemented without the components for creating and maintaining a wireless two-way connection with an external device.
